(19) United States
(12) Patent Application Publication
(10) Pub. No.: US 2016/0354232 A1
(43) Pub. Date: Dec. 8, 2016

(54) THERAPEUTIC COOLING DEVICE AND SYSTEM

(71) Applicant: Renato Rozental, Hartsdale, NY (US)

(72) Inventor: Renato Rozental, Hartsdale, NY (US)

(21) Appl. No.: 15/117,643

(22) PCT Filed: Feb. 9, 2015
(86) PCT No.: PCT/US15/14982

§ 371 Date: Aug. 9, 2016

Related U.S. Application Data
(60) Provisional application No. 61/938,132, filed on Feb. 10, 2014.

(51) Int. Cl. A61F 7/02; A61F 7/10

(52) U.S. Cl. CPC A61F 7/02; A61F 7/10; A61F 2007/0005

(57) ABSTRACT

A cooling pad and system for patient care. The cooling pad includes an upper chamber, a lower chamber, and an intermediate chamber interposed between the upper chamber and the lower chamber. The upper chamber has an internal space and an inlet to accommodate a first cooling medium provided from an external source. The intermediate chamber contains a second cooling medium for transferring hypothermia from the upper chamber to the lower chamber. The lower chamber contains a third cooling medium to deliver hypothermia to the patient's skin. The cooling system can include one or more cooling pads adapted to be positioned on the patient's head and/or neck, and one or more containers for storing the first cooling medium and supplying the first cooling medium to the cooling pad(s).

### CROSS REFERENCE TO RELATED APPLICATION

This application claims priority to U.S. Provisional Application No. 61/938,132, filed Feb. 10, 2014, the disclosure of which is incorporated herein by reference in its entirety.

### FIELD

This invention pertains to a cooling system and device for patient care. More specifically, the invention relates to a cooling pad or pads for ameliorating brain injury and/or spinal cord injury.

### BACKGROUND

The skull is hard and inflexible while the brain is soft with a gelatin-like consistency. The brain is encased inside the skull. During rapid acceleration and de-acceleration the brain moves relative to the skull. Different parts of the brain move at different speeds because of their relative lightness or heaviness. The differential movement of the skull and the brain when the head is struck results in direct brain injury

Brain temperature is higher than core body temperature as much as by 1.5° C. (“core body temperature” refers to a deep internal organ temperature, such as bladder and esophagus). Maintaining a constant basal core temperature, or preventing increase in temperature, following a variety of brain insults is not enough to antagonize the development of long-term lesions. The neuroprotective effects of mild hypothermia (a brain temperature between 33° C. and 36° C.) have been demonstrated in numerous studies. Mild hypothermia is one of the few and most effective neuroprotective therapies against brain ischemia and trauma that currently exists. Preliminary clinical studies have shown that mild hypothermia can be a relatively safe treatment. The feasibility of using mild hypothermia to treat stroke and spinal cord injured patients has been evaluated in some clinical trials. Increasing emphasis is being placed on developing techniques and protocols to ensure rapid cooling of patients.

Often, when a person suffers a head trauma, the neck and spinal column is injured also. The spinal cord also may suffer contusions when the brain is not impacted and needs to be treated separately. As a part of the central nervous system, the tissue of the spinal cord behaves in a similar way to the tissue of the brain when subjected to trauma and contusions can occur. Consequently, similar methods can be used to treat a patient with spinal cord injuries, such as therapeutic hypothermia.

Surface cooling has been used to achieve generalized hypothermia. This sometimes involves submerging the neurosurgical patient in iced water while the patient is on the operating table, and was unwieldy and required prolonged anesthesia. More recently, the use of extracorporeal heat exchanger was explored to treat patients with severe head injuries. Currently, systemic surface cooling using water—circulating blanket is widely used to induce brain hypothermia.

To prevent shivering after heat reduction, a patient treated with systemic cooling often needs be sedated. Other complications that may result from systemic cooling could be promptly handled in a clinical setting but can be difficult to treat outside of a hospital or trauma center because of lack of qualified medical personnel, medical equipment or drugs. Additionally, brain or spinal cord injury resulting from a trauma may be better treated without attempting to cool the entire body.

There is a need for an effective, easy-to-deploy hypothermic apparatus to deliver focal hypothermia (applied only to the head and/or the spinal cord) for use in the field and clinical settings.

### SUMMARY

In one aspect, the present invention provides a cooling pad, which includes an upper chamber having an internal space and at least one inlet to receive a first cooling medium therein, an intermediate chamber disposed adjacent to and in thermal contact with the upper chamber, and a lower chamber disposed adjacent to and in thermal contact with the intermediate chamber. The intermediate chamber includes a second cooling medium, and the lower chamber comprising a third cooling medium.

In some embodiments, the cooling pad includes a plurality of sections adapted to cover the patient's head. In certain embodiments, the cooling pad also includes at least one section configured to cover the patient's neck. In some embodiments, each of the upper, intermediate, and lower chambers of the cooling pad is in the form of a plurality of interconnected cells.

In some embodiments, the upper, intermediate, and lower chambers of the cooling pad are individually sealed and separable from each other. In other embodiments, the upper, intermediate, and lower chambers of the cooling pad form an integral structure, where the upper chamber and the intermediate chamber are separated by a first interface layer, the intermediate chamber and the lower chamber are separated by a second interface layer.

In some embodiments, the second cooling medium has a freezing point lower than the freezing point of the third cooling medium. In one embodiment, the second cooling medium has a freezing point of −10° C. or lower at atmospheric pressure. In some embodiments, the second cooling medium includes a mixture of water and a water soluble polymer. In other embodiments, the second cooling medium includes an ionic liquid. The ionic liquid may include at least one of 1-hexyl-3-methylimidazolium bis(trifluoromethylsulfonyl)imide ([HMIM][Tf2N]) and trihexyl(tetradecyl)phosphonium 2-(tricholoracetyl)pyrrolide. In further embodiments, the second cooling medium comprises an ionic liquid and a polymer soluble in the ionic liquid. The polymer can be a polyelectrolyte.

In some embodiments, the third cooling medium has a freezing point of between about −5° C. and about 5° C. at atmospheric pressure. In one embodiment, the third cooling medium comprises water. In another embodiment, the third cooling medium comprises water and a superabsorbent polymer.

In some embodiments, the inlet of the upper chamber comprises a pressure sensitive bi-directional valve. In further embodiments, the upper chamber can include one or more additional pressure sensitive bi-directional valves for receiving the first cooling medium into the upper chamber or discharging the first cooling medium from the upper chamber.

In some embodiments, the cooling pad includes an outer surface made from a thermally insulating material.

In some embodiments, the cooling pad includes at least one temperature sensor. The temperature sensor may be positioned within one of the upper, intermediate, or lower chambers, or at an interface between the upper chamber and the intermediate chamber, an interface between the intermediate chamber and the lower chamber, or under a lower surface of the lower chamber. In further embodiments, the cooling pad includes a temperature meter operatively coupled with the at least one temperature sensor. The temperature meter includes a circuit for converting signals collected by the temperature sensor to obtain a temperature of the temperature sensor, and a display for indicating the temperature to a user.

In another aspect, the present invention provides a cooling pad without an intermediate chamber interposing between the upper chamber and the lower chamber. The cooling pad includes an upper chamber having at least one inlet to receive a first cooling medium, and a lower chamber disposed in thermal contact with the upper chamber and comprising a third cooling medium. In some embodiments, the third cooling medium can have a freezing point of between about −5° C. and about 5° C. at atmospheric pressure.

In yet another aspect, the present invention provides a cooling system which includes one or more cooling pads described hereinabove, and at least one container configured to store the first cooling medium and providing the first cooling medium into the upper chamber of the cooling pad. In some embodiments, the container is able to withstand a pressure of about 1200 psi to about 4000 psi. In one embodiment, the first cooling medium contained in the container comprises carbon dioxide.

In a further aspect, the present invention provides a method of providing a cooling therapy to a patient. In the method, any of the cooling system and cooling pads described herein can be used. The operator fills the upper chamber of the cooling pad with an amount of the first cooling medium from the container, and positions the cooling pad to cover at least a portion of a patient's head. Positioning the cooling pad can be done before or after filling of the first cooling medium. In some embodiments, the method further includes monitoring the temperature of the lower chamber, and maintaining the temperature of the lower chamber to be between about −35° C. and about 30° C., or between about 10° C. and about 30° C., or between about 0 ° C. and about 4° C.

### BRIEF DESCRIPTION OF THE DRAWINGS

The above and still further features and advantages of the present invention will become apparent upon consideration of the following detailed description of certain specific embodiments thereof, especially when taken in conjunction with the accompanying drawings wherein like reference numerals in the various figures are utilized to designate like components, and wherein:

FIG. 1A is a top view of a cooling pad according to an embodiment of the present invention;

FIG. 1B is a view of the underside of the cooling pad depicted in FIG. 1A;

FIGS. 2A-2C are different schematic views of the cooling pad depicted in FIG. 1A as being positioned on the head and neck of a person or patient;

FIG. 3A is cross sectional view of a cooling pad according to an embodiment of the present invention;

FIG. 3B is cross sectional view of a cooling pad according to another embodiment of the present invention;

FIG. 3C is a cross sectional view of a cooling chamber of an embodiment of the cooling pad of the present invention that has non planar surfaces;

FIG. 3D is a schematic view of a container for storing and supplying a cooling medium for use in a cooling pad according to an embodiment of the present invention;

FIGS. 4A is a schematic exploded view of an embodiment of a cooling pad of the present invention which includes one or more temperature sensors; and

FIG. 4B is a schematic depiction of a temperature meter for measuring a temperature or temperatures as sensed by the temperature sensor(s) included in the cooling pad as depicted in FIG. 4A.

### DETAILED DESCRIPTION

The present invention provides a cooling pad/device and system for cooling a patient's brain, spine, and/or other areas of the body where hypothermia may be beneficial. For example, the device and system of the present invention can be portable and used in the field or clinical settings as part of first aid procedures to provide local hypothermia for traumatic brain or spinal injury, brain ischemia, asphyxia, seizures, or other conditions.

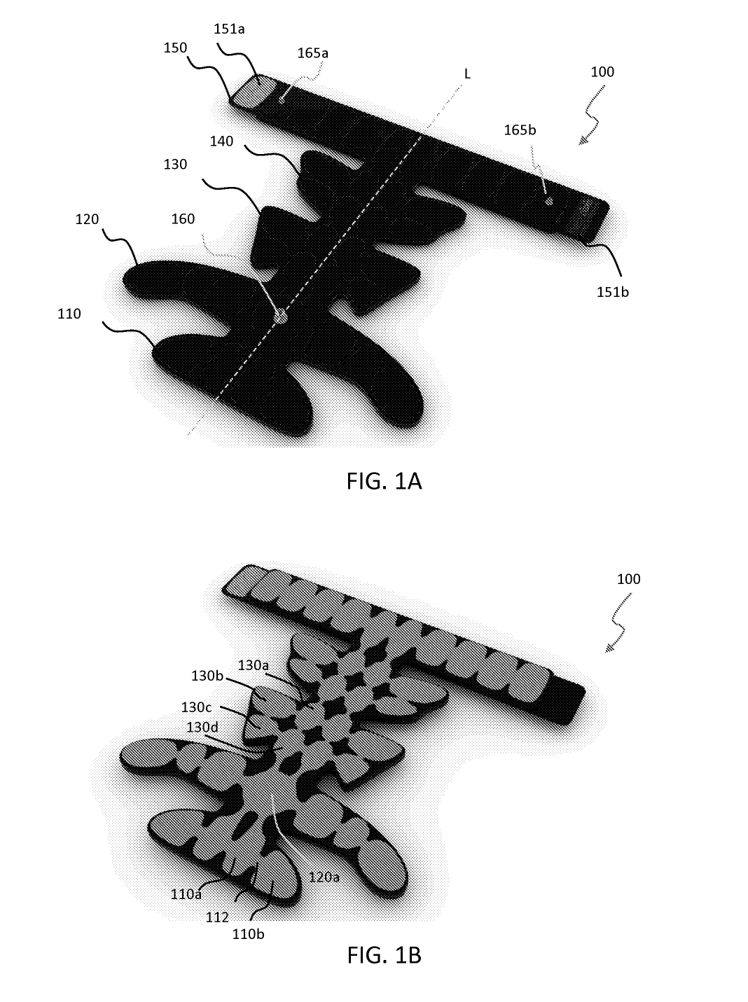

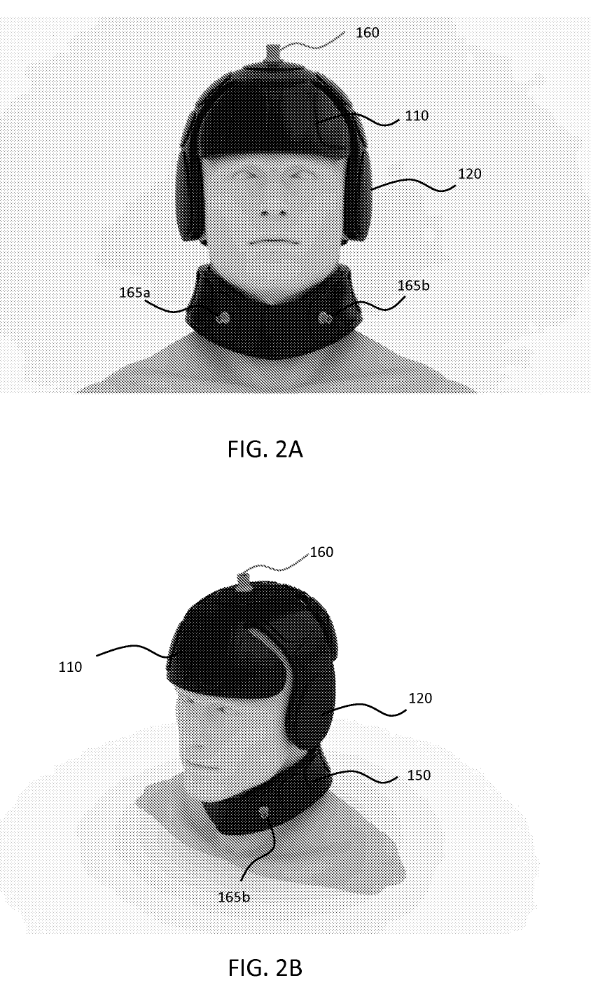

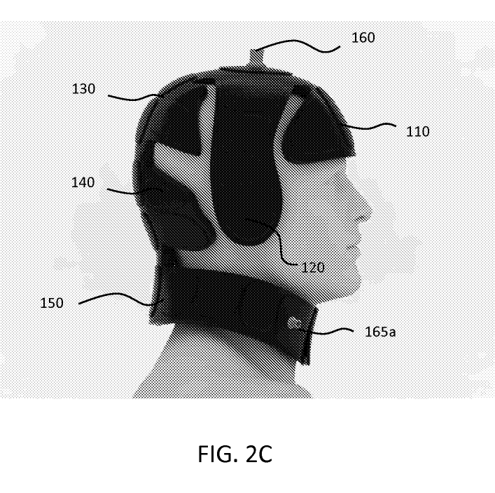

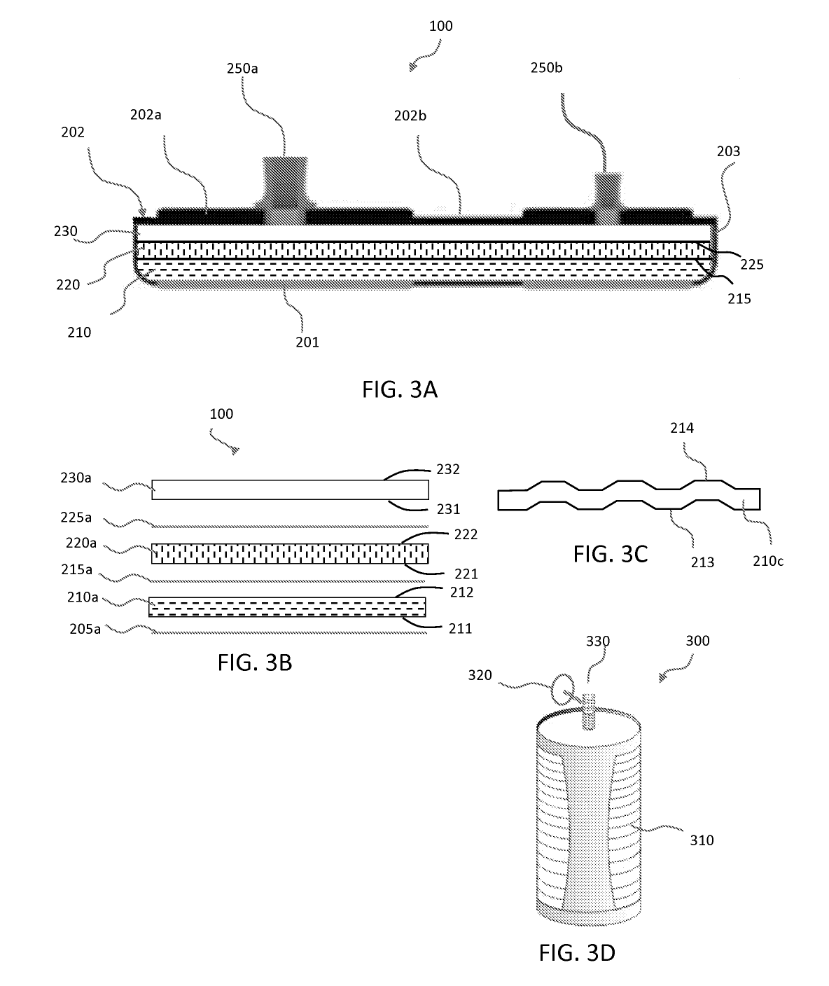

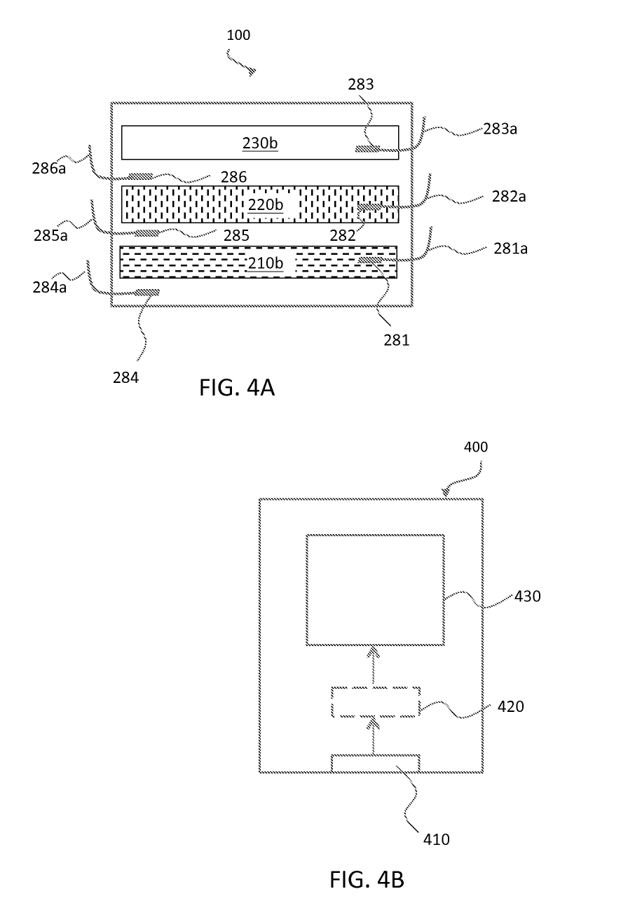

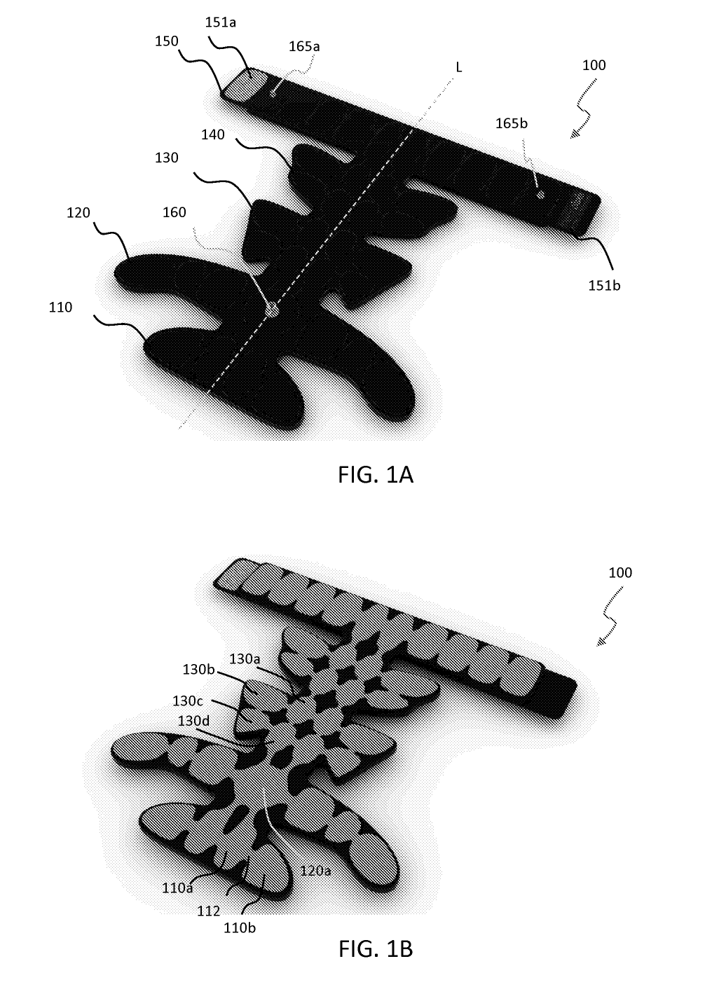

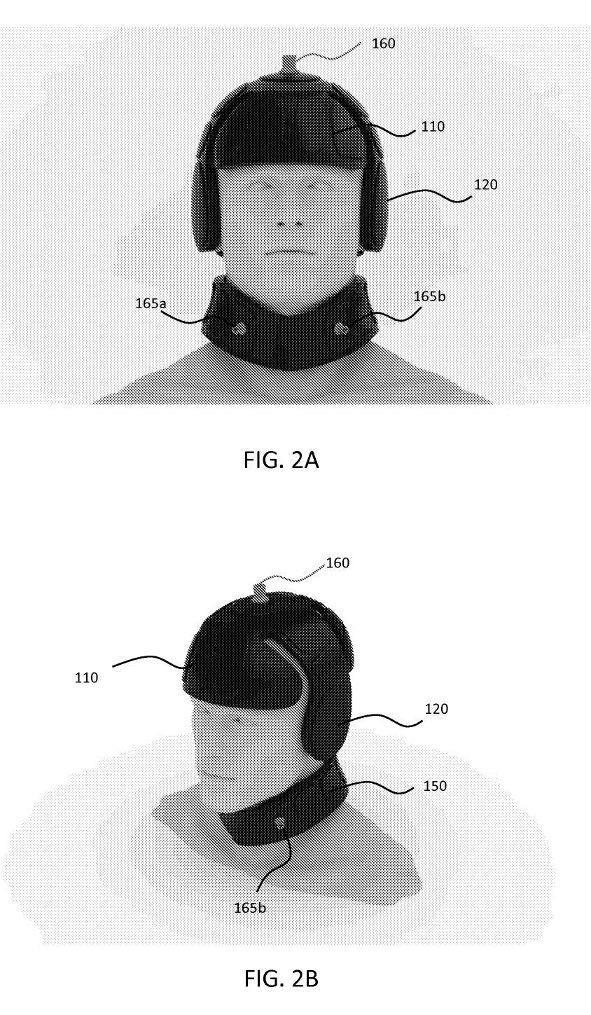

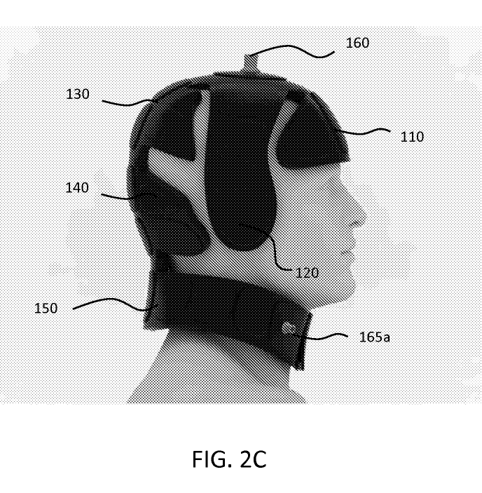

Referring to FIG. 1A, a top view of an embodiment of a cooling pad 100 is illustrated. The pad 100 is substantially symmetrical with respect to a central axis L, and includes a plurality of sections 110, 120, 130, 140, and 150 arranged along the axis. Each of the sections includes two opposing lateral portions extending from the axis, and covers a portion of the patient's head or neck when the pad 100 is deployed on the patient. For example, and as illustrated in FIGS. 2A-, section 110 can cover the patient's forehead and temporal areas, section 120 can cover the central area of the top portion of patient's skull as well as the lateral side extending to the patient's ears, section 130 can cover the upper back portion of the patient's head, section 140 can cover the lower back portion of the patient's head, and section 150 can be wrapped around the patient's neck (this section can cool the cervical portion of the spinal cord). The lateral extensions of section 150 also include fixation elements 151a and 151b near the tips of the extensions for securing the extensions to a person's neck. As illustrated, the fixation elements 151a and 151b can include a pair of Velcro fasteners, with 151a and 151b comprising hooks and loops respectively, or vice versa. Additionally, the cooling pad 100 can include a section or sections that cover the patient's face (not shown), thoracic, lumbar, and lower portion of the spine from the back of the body, etc. The cooling pad 100 can also be configured or adapted to be capable of covering other portions of the body, e.g., an arm, a leg, etc. It is understood that the configuration of these sections of the cooling pad is only illustrative, and alternate arrangements or variations will be apparent to those skilled in the art and therefore encompassed within the scope of the present invention. FIG. 1B is an underside view of the pad 100 depicted in FIG. 1A. As shown, the pad 100 includes a plurality of interconnected cooling cells, some of which on sections 110, 120, and 130 are labeled (110a, 110b, 120a, 130a, 130b, 130c, and 130d). Each of the cells includes an internal space that can accommodate a cooling medium. As will be further explained below, the pad 100 (hence each cells illustrated in FIG. 1B) can include multiple cooling chambers stacked on one another, where within each layer of chamber the cells are in fluidic communication with each other. Also shown in FIG. 1B are narrowed connecting portions between adjacent cells (e.g., connection portion 112 between cells 110a and 110b) to provide greater flexibility for the pad 100 to conform to the patient's head.

Also as shown in FIGS. 1A and 2A-2C, equipped on the cooling pad 100 there are three inlets/outlets 160, 165a and 165b (positioned at the center of section 120, and near the ends of lateral extensions of section 150, respectively). Each of these inlets/outlets can be used to fill a coolant into the upper chamber of the pad 100 for deploying the cooling pad 100 as well as to discharge the coolant from the upper chamber of the cooling pad 100. The positioning of these inlets/outlets as depicted in FIGS. 1A and 2A-2C is merely illustrative and not limiting. Fewer or more inlets/outlets can be included in the cooling pad for filling convenience, temperature distribution control, or to address other operation concerns. For example, the availability of alternative valves avoids the need to move the injured head to implement the cooling mechanism and/or provides optional gas flow rates through the adjustment of the individual bi-directional pressure-sensitive valves.

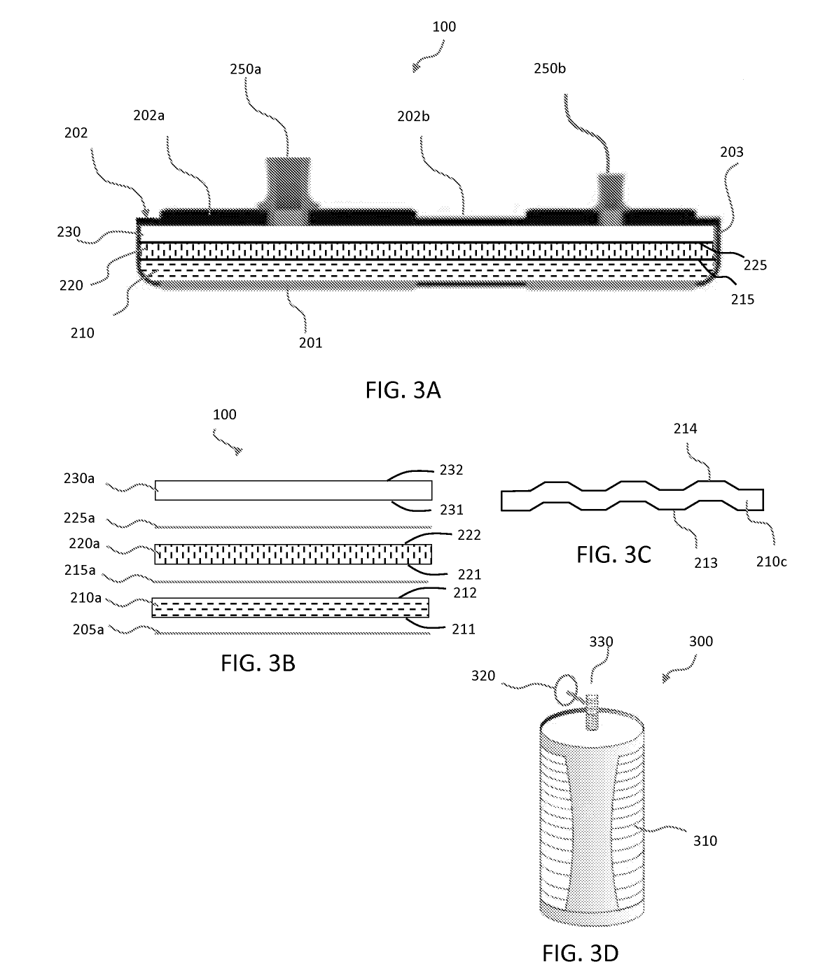

FIG. 3A is a cross sectional view of an embodiment of the cooling pad of the present invention. The cooling pad 100 includes a lower chamber 210, an intermediate chamber 220, and an upper chamber 230. Collectively, chambers 210, 220, and 230 are also referred to as cooling chambers. Each of the cooling chambers 210, 220, and 230 are hermetically sealed (i.e., they are not in fluidic communication with each other). In the embodiment shown in FIG. 3A, the cooling chambers form an integral structure, where the upper chamber 230 and the intermediate chamber 220 are separated by a common interface layer or sheet 225; the intermediate chamber 220 and the lower chamber 210 are separated by a common interface layer or sheet 215. The interface layers 215 and 225 can each comprise a fabric, a nonwoven cellulosic material (such as pressed paper sheets), a polymer film or the like that has good thermal conductance but is impermeable to the coolants to be introduced to the respective cooling chambers.

The cooling pad 100 shown in FIG. 3A also includes an inner surface 201 for contacting the patient's skin (e.g., an area or areas on the patient's head and/or neck), and an outer surface 202 opposite the inner surface 201. The material for the inner surface 201 can be similar to that for the interface layers 215 and 225 as noted above. Additionally, the inner surface can be made from breathable or moisture wicking fabric for the patient's comfort.

The cooling chambers 210, 220, and 230 are sandwiched between the inner surface 201 and the outer surface 202. Additionally, the pad 100 can include a side exterior surface 203 which joins the inner surface 201 and the outer surface 202 to enclose each of the cooling chambers 210, 220, and 230. The side exterior surface 203 may be constructed separately from the outer surface 202 or as an integral extension of the outer surface 202. The outer surface 202 can include portions of different thickness. For example, as shown in FIG. 3A, the portion 202a has a thickness greater than that of portion 202b. The thinner portions can be flexed more easily, thereby facilitating the conformity of the cooling pad to the shape of the skull. The outer surface 202 can comprise or be made from a thermally insulating material, such as a rubbery material that is abrasion-resistant and remains flexible at low temperatures. Example materials include butyl rubber, silicone rubber, neoprene, or other polymeric materials having a glass transition temperature of −10° C. or below, −20° C. or below, or −30° C. or below. Additionally, the outer surface can include thin synthetic-breathable moisture-wicking materials.

FIG. 3B shows an exploded view of an alternative arrangement of the cooling chambers, which include a lower chamber 210a having a lower face 211 and an upper face 212, an intermediate chamber 220a having a lower face 221 and an upper face 222, and an upper chamber 230a having a lower face 231 and an upper face 232. The upper chamber 230a may also include inlets/outlets (not shown) for receiving and/or discharging the first cooling medium. Each of the cooling chambers 210a, 220a, and 230a is a stand-alone sealed structure for accommodating a respective cooling medium therein, and can be directly stacked to form a multilayered structure.

This modular design allows for manufacture flexibility (since the chambers can be fabricated separately and then assembled) and ease of replacement of any of the chambers. Optionally, an interface layer 215a can be disposed between chambers 210a and 220a, and an interface layer 225a can be disposed between chambers 220a and 230a. The lower face 211 of the lower chamber 210a can be used as an inner surface of the pad 100 for contacting the patient's skin. Alternatively, an additional layer 205a can be disposed adjacent the lower face 211 of the lower chamber 210a for contacting the patient's skin. The layer 205a can be made from a material for the inner surface 201 described above in connection with FIG. 2A. The layer 205a may further have openings to allow the hypothermia of the lower chamber 210a to directly flow through the openings and into the patient's skin.

In alternative embodiments, the cooling pad 100 can include an upper chamber 230 (or 230a in FIG. 3B) directly interfacing a lower chamber 210 (or 210a in FIG. 3B), i.e., without including an intermediate chamber interposed between the upper and lower chambers. For such embodiments, the description herein regarding the upper chamber, lower chamber, and other components of the cooling system is applicable.

While shown in FIGS. 3A and 3B each of the cooling chambers has generally planar upper and lower surfaces, any of the cooling chambers may also have non-planar surfaces. As illustrated in FIG. 3C, a cooling chamber 210c can have a non-planar lower surface 213 and/or a non-planar upper surface 214. The nonplanar surfaces can include elevated areas and depressed areas as shown, which can be patterned as desired. When the contacting surfaces between two adjacent cooling chambers are non-planar, it is preferable to have surface elevations and depressions on the two surfaces in a mating configuration so as to maximize the contacting area for exchange thermal energy between the two chambers. If the lower cooling chamber has a non-planar lower surface, only a portion of the lower surface directly contacts the patient's skin.

Referring back to FIG. 3A, before the cooling pad is deployed for use on a patient, the upper chamber 230 (or 230a in FIG. 3B) of the cooling pad 100 has a hollow interior space to accommodate a first cooling medium, and includes an inlet 250a for filling an amount of the first cooling medium into the upper chamber 230 from an external source. The first cooling medium may have a freezing point at atmospheric pressure (i.e., 1 atm) of −70° C. or lower, −100° C. or lower, −150° C. or lower, or −200° C. or lower. The first cooling medium after entering the upper chamber 230 may be a gas, a gas/liquid mixture, a gas/solid mixture, or a liquid for a period of time. For example, it can be CO₂ gas, a mixture of CO₂ gas and dry ice pellets, and/or nitrogen gas. The upper chamber 230 is also equipped with an outlet 250b for discharging the first cooling medium. Each of inlet 250a and outlet 250b can include a bi-directional valve having a threshold pressure that is preset or manually adjustable for controlling the amount of cooling medium in the upper chamber 230. The threshold can be greater than the atmospheric pressure, e.g., about 10% to about 1000% greater than the atmospheric pressure, or about 20% to about 100% greater than the atmospheric pressure. In some embodiments, the valve may also be opened manually. The use of different first cooling media can achieve (at a rate of about 0.1° C. to about 0.5° C./hour) mild (about 36° C.) or moderate (about 33° C. to about 35° C.) brain hypothermia, which is about 2.5° C. to about 4.5° C. below the normal range of brain temperature which ranges from about 37.5° C. to about 38.0° C.

For use with the cooling pad, one or more containers for storing and supplying the first cooling medium is also provided, e.g., as part of a cooling system or kit. The cooling system can also include a helmet, such as a military helmet, a civil helmet (e.g., for engineering, construction, sports, and other uses) in which the cooling pad can be fitted or secured, e.g., by securing mechanisms located on the interior of the helmet. The containers may be portable and/or handheld. As illustrated in FIG. 3D, a portable container 300 can have a body 310 in a form of a cylinder or canister. The container may be a metallic (e.g., aluminum) canister that can withstand an internal pressure in excess of 100 psi, 200 psi, 500 psi, 1000 psi, 2000 psi, or 4000 psi. In some embodiments, the container can withstand an internal pressure of about 1200 psi to about 4000 psi. When the container is activated, the first cooling medium will flow out from the release nozzle 330. The release nozzle 330 may be designed to couple with the inlet(s) of the cooling pad in an airtight manner (e.g., directly by a threaded connection or through a segment of tubing). The operator may be able to observe and adjust the rate of the coolant flow into the cooling pad through a flow meter 320 coupled to the release nozzle 330. The first cooling medium stored in the container can include liquid CO₂, liquid nitrogen, or other cryogenic fluids.

When the first cooling medium enters the upper chamber of the cooling pad, a portion may phase change into a gas due to the dramatic reduction of pressure in the upper chamber as compared to the pressurized container where the first cooling medium is originally stored. In operation, by adjusting parameters such as the rate of filling, the pressure threshold of the bi-directional valves, the first cooling medium can bring the temperature of the upper chamber 230 to a temperature of −30° C., −40° C., −50° C., −60° C., −70° C. or even lower. In some embodiments, during operation (when the cooling pad is positioned on a patient to cool the patient's head), the temperature of the upper chamber can be maintained in a range between about −30° C. and about −15° C. By thermal conductance of the intermediate and lower chambers which mediate the coldness felt by the patient, the cooling pad and system can be used to achieve the desired cooling for patients in a setting (e.g., a war zone, a desert) where long term storage of ice packs is unwieldy or impossible, for treating patients with head injuries in emergencies.

The intermediate chamber 220 (or 220a in FIG. 3B) of the cooling pad 100 includes a second cooling medium, which can be prefilled into the chamber when the cooling pad is manufactured and before use. The intermediate chamber acts as a reservoir to maintain a desired temperature of the lower chamber for a prolonged period of time. In some embodiments, the construction and cooling medium for the intermediate chamber are such that the intermediate chamber is capable of maintaining the temperature of the lower chamber at a desired temperature, e.g., about 0° C., for a desired or predetermined amount of time, e.g., about 2-8 hours (for example, about 3 hours, about 4 hours, about 5 hours, or about 6 hours). In some embodiments, the second cooling medium has a freezing point of about −10° C. or lower at atmospheric pressure. In some embodiments, the second cooling medium has a freezing point of −20° C. or lower, or −30° C. or lower. In some embodiments, the second cooling medium can have a freezing point of from about −40° C. to about 0° C., or from about −30° C. to about −5° C., or from about −20° C. to −10° C.

In some embodiments, the second cooling medium can include water and an agent that reduces the freezing point of water, such as polyethylene glycol, or other nontoxic anti-icing or anti-freezing agent. In other embodiments, the intermediate chamber can be filled with an ionic liquid. Ionic liquids as green solvents have been studied extensively recently, thanks to their properties as low vapor pressure, high thermal stability, and ability to solvate compounds of widely varying polarity. An ionic liquid can contain cations and anions, where the cations can include but are not limited to variously substituted imidazolium salts, as well as ammonium, pyridinium, isoquinolinium, sulfonium, phosphonium, pyrrolidinium, and other complex compounds, and the anions can include but are not limited to nitrite, nitrate, sulfate, tosylate, phosphate, acetate, and various fluoro and boron containing compounds, such as tetrafluoroborate, tetraphenylborate, tetrakis-((4-trifluoromethyl)phenyl)borate, bis(2-methyllactato)borate, perfluoroethylimide, bis((trifluoromethyl)sulfonyl)imides, hexafluorophosphate, alkylcarbonicosahedral, etc. In exemplary embodiments, the ionic liquid can include 1-hexyl-3-methylimidazolium bis(trifluoromethylsulfonyl)imide ([hMIM][Tf2N]) and trihexyl(tetradecyl)phosphonium 2-(tricholoracetyl)pyrrolide. In further embodiments, the second cooling medium can include an ionic liquid as well as a polymer soluble in the ionic liquid, such as a polyelectrolyte, e.g., sodium polyacrylate.

The lower chamber 210 (or 210a in FIG. 3B) of the cooling pad 100 can include a third cooling medium. The third cooling medium can be prefilled into the lower chamber when the cooling pad is manufactured and before use. In some embodiments, the third cooling medium can have a freezing point higher than the freezing point of the second cooling medium at atmospheric pressure. For example, the freezing point of the third cooling medium can be from about 5° C. to about 20° C. higher than the freezing point of the second cooling medium. In some embodiments, the freezing point of the third cooling medium can be from about −5° C. to about 5° C. at atmospheric pressure. In some embodiments, the third cooling medium has a freezing point of from about −2° C. to about 2° C. at atmospheric pressure.

In some embodiments, the third cooling medium can be water. In other embodiments, the third cooling medium can include water and an additive. For example, the additive can include a water soluble polymer such as a superabsorbent polymer (a polymer that can absorb at least 100 times of water relative to own weight). The additive can also include non-soluble inorganic materials, such as graphite, silica, clay, glass fibers, or the like, as well as surfactants, salts, alcohols, etc.

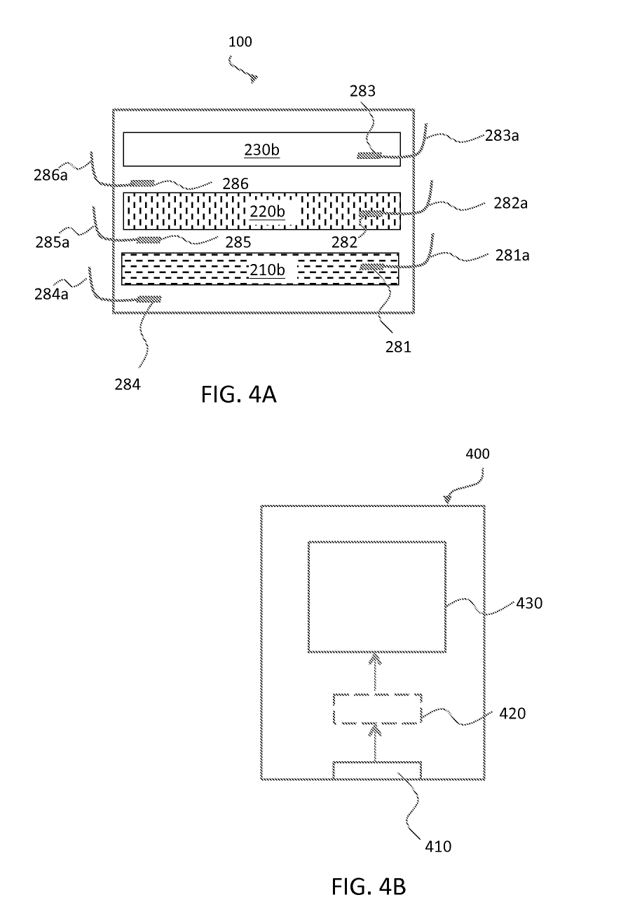

In some embodiments, the cooling pad of the present invention can include temperature measuring components. For example, one (and any) or more of the cooling chambers can include or embed one or more temperature sensors. As illustrated in FIG. 4A, the cooling pad 100 may include a temperature sensor 281 in the lower chamber 210b, and/or a temperature sensor 282 in the intermediate chamber 220b, and/or a temperature sensor 283 in the upper chamber 230b. Each of the temperature sensors 281, 282 and 283 can be coupled with a wire or lead 281a, 282a, and 283a, respectively, for transmitting the signals sensed by the sensors. Alternatively or additionally, the cooling pad 100 may include a temperature sensor 284 disposed under the lower chamber 210b, and/or a temperature sensor 285 disposed at an interface between the intermediate chamber 220b and the lower chamber 210b, and/or a temperature sensor 286 disposed at an interface between the upper chamber 230b and the intermediate chamber 220b. Each of the temperature sensors 284, 285 and 286 can be coupled with a wire or lead 284a, 285a, and 286a, respectively, for transmitting the signals sensed by the sensors. The wires or leads 281a, 282a, 283a, 284a, 285a, and 286a can extend out of the cooling pad 100 for connection with a temperature meter.

FIG. 4B schematically depicts a temperature meter 400 that can take input from any (or all) of the temperature sensors 281-286 shown in FIG. 4A. The temperature meter 400 includes a signal input port (or ports) 410 to receive any or all of the leads 281a-286a, and a circuit 420 for converting the signals sensed by the temperature sensor(s) and transmitted by the leads, and obtaining the temperature(s) of the temperature sensor(s). The circuit 420 sends the temperature(s) to display to the user on a display 430 (e.g., a LCD display). When more than one temperature sensor is used, the display 430 can simultaneously display multiple temperatures for different portions of the cooling pad. In alternative embodiments, the temperature sensors can include modules that transmit signals wirelessly to the temperature meter, which is equipped with a wireless receiver to receive the transferred signals. In such a system, the leads or wires 281a-286a in FIG. 4A are not required.

To use the cooling pad of the present invention for cooling a patient's head, a user or operator can first charge the upper chamber with an amount of the first cooling medium using the container containing the first cooling medium, and then position the cooling pad on the patient to cover the desired portions of the patient's head. Alternatively, the user can first position the cooling pad on the patient's head and then charge the first cooling medium into the upper chamber. When the cooling pad is in use on a patient, the temperatures of different portions of the cooling pad can be actively monitored to ensure proper functioning of the pad. For example, the user can monitor the temperature of the lower chamber, e.g., by using one or more temperature sensors embedded in the lower chamber, and maintain the lower chamber at a temperature between about −35° C. and about 30° C., for example, between about −10° C. and about 10° C., between about −4° C. and about 20° C., between about 0° C. and about 4° C., between about −5° C. and about 5° C., or between −2° C. and about 2° C., for the duration of the treatment or any portion thereof. The suitable temperature ranges for the lower chamber for each patient may be different depending on the patient's condition, age, as well as the specifics or extent of the head and/or spinal cord injury. For example, for neonatal use or when used to treat infants, in some embodiments, the lower chamber can be maintained between about 10° C. and about 30° C., or between about 15° C. and about 25° C. when the cooling pad is in use. For adult patients, in some embodiments, the lower chamber may be maintained between about 0° C. and about 4° C. In some embodiments, the cooling medium in the lower chamber is maintained at its freezing point or slightly below the freezing point (e.g., about 5 degrees, or about 2 degrees below the freezing point). Alternatively, the operator can monitor the temperature of the interface between the lower chamber and the patient's skin, e.g., by using a temperature sensor positioned at such an interface (e.g., sensor 284 illustrated in FIG. 4A), and maintain such temperature within a desired range, e.g., from about −2° C. to about 2° C., or from about 0° C. to about 4° C., etc. Again, the suitable ranges for this temperature for each patient may be different depending on the patient's condition, age, as well as the specifics or extent of the head and/or spinal cord injury. To adjust the temperature, in some embodiments, the user can fill in more cooling medium into the upper chamber (when the temperature is above the desired value), or release a portion of the cooling medium in the upper chamber by opening one or more of the inlets/outlets of the upper chamber (when the temperature is below the desired value). Additionally, if the temperature is too low, the operator may also temporarily remove the cooling pad from the patient's head.

Alternatively or additionally, the user can monitor the temperature of the intermediate chamber, e.g., by using one or more temperature sensors embedded in the intermediate chamber. In some embodiments, the temperature of the intermediate chamber can be maintained at between about −30° C. and about −5° C. In other embodiments, the temperature of the intermediate chamber can be maintained between about −20° C. and about −10° C. In further embodiments, the temperature of the intermediate chamber can be maintained at above the freezing temperature of the second cooling medium, e.g., from about 5° C. to 10° C. above the freezing temperature of the second cooling medium. In other embodiments, the temperature of the intermediate chamber can be maintained at below the freezing temperature of the second cooling medium.

In addition to temperature sensors, the cooling pad and cooling system of the present invention can further include other sensors, such as blood pressure sensors, electroencephalography (EEG) sensors or electrodes, and other sensors that detect and/or measure the patient's physiological conditions, such as posture, movement, breath, heart pulse frequency, etc. Such sensors can be attached to the cooling pad, e.g., at the underside that contact the patient's skin, and positioned as appropriate on the patient's head or neck. Signals from such sensors can be sent through wired or wireless connection to suitable monitoring devices.

By adjusting operating parameters of the cooling pad of the present invention, which include but are not limited to the type of the first cooling medium, the amount of the first cooling medium to fill in the upper chamber, the pressure threshold of the one or more bidirectional valves of the upper chamber, the cooling pad of the present invention can cool the patient's brain to a mean temperature of between about 33° C. and about 36° C. within about 24 hours and can maintain the temperature of the patient's brain at such temperature range for about 24 hours to about 96 hours.

The cooling pad of the brain cooling system can be used to cool the brain at a controlled rate over a specific amount of time to a specific mean temperature. As used herein, the term controlled may mean constant, i.e., does not vary over time where the time period can be controlled to be as short or as long as needed. Overall, different controlled rates may be used with the same patient. The rate of cooling may be linear or non-linear.

The time required to meet a mean temperature in the brain of about 33° C. may range from about 12 hours to about 18 hours. The mean temperature may be achieved using the cooling pad, or alternatively using the cooling pad in conjunction with advanced medical facilities available in hospitals. Other higher mean temperatures in the brain may be achieved in shorter time periods ranging from immediately after the insult to the brain to about 2 hours depending on the rate of cooling. An intravenous saline solution which is maintained at temperatures ranging from about 4° C. to about 5° C. in quantities such as 0.5, 1.0 and 1.5 liters may be provided to a patient to aid in cooling of the brain.

The mean temperature of the brain after hypothermia induction will usually be lower than the core body temperature. The mean temperature of the brain after hypothermia induction may range from about 33° C. to about 36° C., from about 34° C. to about 37° C., from about 33.5° C. to about 36.5° C., from about 34° C. to about 36° C., from about 35° C. to about 36° C., from about 32° C. to about 35° C. or from about 32° C. to about 33° C.

The mean temperature of the brain may be maintained for an extended period such as about 24 hours to about 96 hours, about 36 hours to about 72 hours, about 48 hours to about 56 hours, or about 48 hours. The temperature may be maintained using the cooling pad or alternatively the cooling pad in conjunction with advanced medical facilities.

The sensitivity, i.e., the resultant temperature change, and/or the resultant rate of temperature change, experienced by the patient, will depend on the physical conditions of the patient, e.g., the size and age of the patient. Furthermore, calculations can be done to determine how cold the head might become if all the cooling is focused solely in the head. The amount of cooling to the head can be calculated using the following assumptions: (1) mass of brain, for example, 1.4 kg, (2) specific heat of water and (3) heat transfer from body (warming from cerebral blood flow) is negligible. Heat load calculation is an important part of sizing and designing a radiant heating/cooling system. There are two types of heat loss to consider: conduction and convection.

Calculations—Calculate ΔT For example, ΔT is a difference between brain core temperature (38° C.) and brain surface temperature (37.5° C.). ΔT=0.5° C. Brain weight: 1.4 Kg (75% water), Blood flow: 1.25 liters/min, Brain volume: 1,400 cc (cm³).

A typical brain heat load calculation consists of surface heat loss calculation through convection and heat loss due to blood flow (i.e., conduction). The cooling pad modulates the extent of heat loss mainly by conduction. AT can be calculated using the Fourier law:

$${q}^{″} = \frac{q}{A} = - k \frac{\partial T}{\partial x}$$

taking in consideration the physical “barriers” which slow down or resist heat transfer from the brain (e.g., empty spaces between the head and the cooling pad). Brain heat loss vs. rewarming by systemic blood flow (37° C.): The mass of circulating blood within the brain per minute is similar to the brain mass. The amount of heat to be removed from the brain in order to drop in 1° C. the brain temperature:

Q=mcΔT
m=1.4 kg
c=1 kcal/kg/C (considering specific heat of water).
   Thus, it will take 1400 calories for each 1° C. drop. Energy provided by brain blood flow: Considering 1.25 liters/min, AT of 1° C. and 30 min of perfusion (i.e., within 30 min.about.37 liters or 37 Kg)—there is a need of 37,500 calories for each 1° C. drop--or a continuously removal of 75 kcal/h to drop the blood temperature in 1° C.

The brain may then be warmed at a rate ranging from about 0.1° C. to about 0.3° C./hour, about 0.1° C. to about 0.2° C./hour or about 0.2° C. to about 0.3° C./hour. The time required to re-warm the brain may range from about 24 hours to about 96 hours, about 36 hours to about 72 hours, about 48 hours to about 56 hours, or about 48 hours. Re-warming of the brain can be handled in a clinical setting.

As used herein, the term “about” when used to refer to a temperature value means a range within ±1° C. deviation from the given temperature value, and when used to refer to a duration of time or other quantities means a deviation of up to ±10% from the given value.

While illustrative embodiments of the invention have been disclosed herein, numerous modifications and other embodiments may be devised by those skilled in the art in accordance with the invention. For example, it is appreciated that the cooling pad as described herein can also be used to provide hypothermic therapy to other parts of the body, such as the back, an arm, a leg, a foot, etc., and for other conditions of the patient where hypothermia may be beneficial. Therefore, it will be understood that the appended claims are intended to include such modifications and embodiments, which are within the spirit and scope of the present invention.

What is claimed is:

1. A cooling pad, comprising:
an upper chamber having an internal space and at least one inlet to receive a first cooling medium therein,
an intermediate chamber disposed adjacent to and in thermal contact with the upper chamber, the intermediate chamber comprising a second cooling medium; and
a lower chamber disposed adjacent to and in thermal contact with the intermediate chamber, the lower chamber comprising a third cooling medium.

2. The cooling pad of claim 1, wherein the cooling pad includes a plurality of sections adapted to cover the patient's head.

3. The cooling pad of claim 2, further comprising at least one section configured to cover the patient's neck.

4. The cooling pad of claim 1, wherein the upper, intermediate, and lower chambers are individually sealed and separable from each other.

5. The cooling pad of claim 1, wherein the upper, intermediate, and lower chambers form an integral structure, and wherein the upper chamber and the intermediate chamber are separated by a first interface layer, the intermediate chamber and the lower chamber are separated by a second interface layer.

6. The cooling pad of claim 1, wherein each of the upper, intermediate, and lower chambers is in the form of a plurality of interconnected cells.

7. The cooling pad of claim 1, wherein the second cooling medium has a freezing point at atmospheric pressure of −10° C. or lower.

8. The cooling pad of claim 1, wherein the second cooling medium comprises a mixture of water and a water soluble polymer.

9. The cooling pad of claim 1, wherein the second cooling medium comprises an ionic liquid.

10. The cooling pad of claim 9, wherein the ionic liquid comprises at least one of 1-hexyl-3-methylimidazolium bis(trifluoromethylsulfonyl)imide ([HMIM][Tf2N]) and trihexyl(tetradecyl)phosphonium 2-(tricholoracetyl)pyrrolide.

11. The cooling pad of claim 9, further comprising a polymer soluble in the ionic liquid.

12. The cooling pad of claim 11, wherein the polymer is a polyelectrolyte.

13. The cooing pad of claim 1, wherein the third cooling medium has a freezing point of between about −5° C. and about 5° C. at atmospheric pressure.

14. The cooling pad of claim 1, wherein the third cooling medium comprises water.

15. The cooling pad of claim 14, wherein the third cooling medium comprises a superabsorbent polymer.

16. The cooling pad of claim 1, wherein the inlet of the upper chamber comprises a pressure sensitive bi-directional valve.

17. The cooling pad of claim 16, wherein the upper chamber comprises one or more additional pressure sensitive bi-directional valves for receiving the first cooling medium into the upper chamber or discharging the first cooling medium from the upper chamber.

18. The cooling pad of claim 1, further comprising an outer surface made from a thermally insulating material.

19. The cooling pad of claim 1, further comprising at least one temperature sensor.

20. The cooling pad of claim 19, wherein the at least one temperature sensor is positioned within one of the upper, intermediate, or lower chambers, or at an interface between the upper chamber and the intermediate chamber, an interface between the intermediate chamber and the lower chamber, or under a lower surface of the lower chamber.

21. The cooling pad of any of claim 19 or claim 20, further comprising a temperature meter operatively coupled with the at least one temperature sensor, the temperature meter comprising:
a circuit for converting signals collected by the temperature sensor to obtain a temperature of the temperature sensor; and
a display for indicating the temperature to a user.

22. A cooling pad, comprising:
an upper chamber having at least one inlet to receive a first cooling medium; and
a lower chamber disposed in thermal contact with the upper chamber, the lower chamber comprising a third cooling medium.

23. The cooling pad of claim 22, wherein the third cooling medium has a freezing point of between about −5° C. and about 5° C. at atmospheric pressure.

24. A cooling system for patient care, comprising:
a cooling pad of any of claims 1-23; and
at least one container configured to store the first cooling medium and providing the first cooling medium into the upper chamber of the cooling pad.

25. The cooling system of claim 24, wherein the first cooling medium comprises carbon dioxide.

26. A method of providing a cooling therapy to a patient, comprising:
providing a cooling system of claim 24;
filling the upper chamber of the cooling pad with an amount of the first cooling medium from the container; and
position the cooling pad to cover at least a portion of a patient's head.

27. The method of claim 26, further comprising:
monitoring the temperature of the lower chamber, and maintaining the temperature of the lower chamber to be between about −35° C. and about 30° C.

28. The method of claim 26, further comprising:
monitoring the temperature of the lower chamber, and maintaining the temperature of the lower chamber to be between about 10° C. and about 30° C.

29. The method of claim 26, further comprising:
monitoring the temperature of the lower chamber, and maintaining the temperature of the lower chamber to be between about 0° C. and about 4° C.
